(19) United States
(12) Patent Application Publication
(10) Pub. No.: US 2023/0404917 A1
(43) Pub. Date: Dec. 21, 2023

(54) RUXOLITINIB OR DEUTERATED RUXOLITINIB COMPOSITION AND USES THEREOF

(71) Applicant: Sol-Gel Technologies Ltd., Ness Ziona (IL)

(72) Inventors: Moshe ARKIN, Kfar Shmaryahu (IL); Ofer TOLEDANO, Kfar Saba (IL); Karine NEIMANN, Ness Ziona (IL)

(73) Assignee: Sol-Gel Technologies Ltd., Ness Ziona (IL)

(21) Appl. No.: 18/037,568

(22) PCT Filed: Nov. 17, 2021
(86) PCT No.: PCT/IL2021/051362

§ 371 Date: May 18, 2023

Related U.S. Application Data
(60) Provisional application No. 63/115,745, filed on Nov. 19, 2020.

(51) Int. Cl. A61K 9/06; A61K 9/00; A61K 31/519; A61K 45/06

(52) U.S. Cl. CPC A61K 9/06; A61K 9/0014; A61K 31/519; A61K 45/06

(57) ABSTRACT

This invention, in some embodiments thereof, relates to regimens, topical compositions comprising ruxolitinib, deuterated ruxolitinib or pharmaceutically acceptable salt thereof and uses thereof for the treatment of inflammatory skin conditions.

### FIELD OF THE INVENTION

This invention, in some embodiments thereof, relates to regimens, topical compositions comprising ruxolitinib, deuterated ruxolitinib or pharmaceutically acceptable salt thereof and uses thereof for the treatment of inflammatory skin conditions.

### BACKGROUND OF THE INVENTION

Janus kinase inhibitors, also known as JAK inhibitors or jakinibs (henceforth JAK inhibitors or JAKi), are a class of drugs interfering with the JAK-STAT signaling pathway by inhibiting at least one of the Janus kinase enzymes JAK1, JAK2, JAK3 or TYK2. Some JAK inhibitors inhibit all the above enzymes and are therefore named pan-JAK inhibitors. Ruxolitinib is one example of a JAK inhibitor.

Ruxolitinib therapy (systemic) is often associated with adverse events of thrombocytopenia (low platelet count) and anemia (low hemoglobin). Thrombocytopenia is dose dependent and is considered a dose limiting toxic effect.

Atopic dermatitis (AD) is a highly pruritic chronic inflammatory skin disorder.

Ruxolitinib, a selective inhibitor of Janus kinase 1 and Janus kinase 2, potently suppresses cytokine signaling involved in AD pathogenesis.

There is a need for long-term treatment of inflammatory skin conditions, their symptoms and associated conditions in a safe and effective manner, for example by topical administration of ruxolitinib compositions.

### SUMMARY OF THE INVENTION

This invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier.

This invention further provides a method of treatment, prevention or alleviation of an inflammatory skin condition, wherein the method comprises administering to a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier.

This invention further provides a regimen for the treatment, prevention or alleviation of an inflammatory skin condition comprising topically applying onto an affected skin area of a subject in need thereof, at least once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier. In other embodiments, the topical composition is administered once daily, twice daily or three times per day.

This invention further provides a method of treatment, prevention or alleviation of an inflammatory skin condition, wherein the method comprises administering to a subject in need thereof, at least once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption. In other embodiments, the topical composition is administered once daily, twice daily or three times per day.

### DETAILED DESCRIPTION OF THE INVENTION

This invention provides a regimen and methods for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier. The regimen and methods of this invention provide comparable or lower side effects compared to systemic administration or topical administration of a corresponding ruxolitinib, deuterated ruxolitinib, or pharmaceutically acceptable salt thereof composition (in terms of AUC) known in the art.

### Compositions

This invention provides and makes use, in one aspect, of a topical composition comprising more than 1.5% w/w ruxolitinib or pharmaceutically acceptable salt thereof, or deuterated ruxolitinib or a pharmaceutically acceptable salt thereof and a pharmaceutically acceptable carrier. In one embodiment, the ruxolitinib or pharmaceutically acceptable salt thereof, or deuterated ruxolitinib or a pharmaceutically acceptable salt thereof is in an amount of more than 1.5% to about 5.0% w/w. In another embodiment, ruxolitinib or pharmaceutically acceptable salt thereof, or deuterated ruxolitinib or a pharmaceutically acceptable salt thereof is in an amount of 1.6% to about 3.0% w/w. In another embodiment, ruxolitinib or pharmaceutically acceptable salt thereof, or deuterated ruxolitinib or a pharmaceutically acceptable salt thereof is in an amount of 2% to about 3.0% w/w. In another embodiment, the ruxolitinib or pharmaceutically acceptable salt thereof, or deuterated ruxolitinib or a pharmaceutically acceptable salt thereof is in an amount of between about 2.0% to about 2.5% w/w. In another embodiment, the ruxolitinib or pharmaceutically acceptable salt thereof, or deuterated ruxolitinib or a pharmaceutically acceptable salt thereof is in an amount of about 2.5% w/w. In another embodiment, the amount is of between more than 1.5% and 2.0% w/w. In another embodiment, the amount is of between about 2.5% and 3.0% w/w. In another embodiment, the amount is of between about 3.0% and 3.5% w/w. In another embodiment, the amount is of between about 3.5% and 4.0% w/w. In another embodiment, the amount is of between about 4.0 and 4.5% w/w. In another embodiment, the amount is of between about 4.5% and 5.0% w/w. In another embodiment, the amount is 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% w/w. Each possibility represents a separate embodiment of this invention.

In some embodiments, the topical composition of this invention comprises ruxolitinib an amount of more than 1.5% to about 5.0% w/w and a pharmaceutically acceptable salt. In some embodiments, the topical composition of this invention comprises ruxolitinib an amount of 1.6% to about 5.0% w/w and a pharmaceutically acceptable salt.

In some embodiments, the topical composition of this invention comprises a deuterated ruxolitinib in an amount of more than 1.5% to about 5.0% w/w and a pharmaceutically acceptable salt. In some embodiments, the topical composition of this invention comprises a deuterated ruxolitinib in an amount of 1.6% to about 5.0% w/w and a pharmaceutically acceptable salt.

In some embodiments, the topical composition of this invention comprises ruxolitinib phosphate in an amount of more than 1.5% to about 5.0% w/w.

In some embodiments, non-limiting examples of pharmaceutically acceptable salts of ruxolitinib or deuterated ruxolitinib within the compositions of this invention include: chloride, acetate, citrate, lactate, mesylate, nitrate, sulfate, phosphate, diphosphate, tartrate, carbonate and bicarbonate. Each possibility represents a separate embodiment of this invention. In one embodiment, the salt is phosphate.

This invention provides and makes use, in one aspect, of a topical composition comprising more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

In another embodiment, provided herein, a topical composition comprising more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability.

In another embodiment, provided herein, a topical composition comprising more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has no systemic absorption.

In another embodiment, provided herein, a topical composition comprising more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has low systemic absorption.

In another embodiment, the ruxolitinib or pharmaceutically acceptable salt thereof, or deuterated ruxolitinib or a pharmaceutically acceptable salt thereof is in an amount of more than 0.5% to about 5.0% w/w. In another embodiment, ruxolitinib or pharmaceutically acceptable salt thereof, or deuterated ruxolitinib or a pharmaceutically acceptable salt thereof is in an amount of 0.6% to about 5% w/w. In another embodiment, ruxolitinib or pharmaceutically acceptable salt thereof, or deuterated ruxolitinib or a pharmaceutically acceptable salt thereof is in an amount of 0.6% to about 1.5% w/w. In another embodiment, the ruxolitinib or pharmaceutically acceptable salt thereof, or deuterated ruxolitinib or a pharmaceutically acceptable salt thereof is in an amount of between about 1.5% to about 2.5% w/w. In another embodiment, the ruxolitinib or pharmaceutically acceptable salt thereof, or deuterated ruxolitinib or a pharmaceutically acceptable salt thereof is in an amount of about 2.5% w/w. In another embodiment, the amount is of between more than 1.5% and 2.0% w/w. In another embodiment, the amount is of between about 2.5% and 3.0% w/w. In another embodiment, the amount is of between about 3.0% and 3.5% w/w. In another embodiment, the amount is of between about 3.5% and 4.0% w/w. In another embodiment, the amount is of between about 4.0 and 4.5% w/w. In another embodiment, the amount is of between about 4.5% and 5.0% w/w. In another embodiment, the amount is 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% w/w. Each possibility represents a separate embodiment of this invention.

In some embodiments, the topical composition of this invention comprises ruxolitinib an amount of more than 0.5% to about 5.0% w/w and a pharmaceutically acceptable salt, and wherein the composition has decreased skin permeability, no systemic absorption or low systemic absorption.

In some embodiments, the topical composition of this invention comprises a deuterated ruxolitinib in an amount of more than 0.5% to about 5.0% w/w and a pharmaceutically acceptable salt, and wherein the composition has decreased skin permeability, no systemic absorption, or low systemic absorption.

In some embodiments, the topical composition of this invention comprises ruxolitinib phosphate in an amount of more than 0.5% to about 5.0% w/w, and wherein the composition has decreased skin permeability, no systemic absorption or low systemic absorption.

In one embodiment, this invention provides a dosage form comprising the compositions as described hereinabove, wherein the composition is formulated as a cream, a gel, an ointment, an emulsion, a solution, a suspension, an elixir, a lotion, a tincture, a paste, a foam, an aerosol, a spray, a patch, a transdermal patch or an applicator syringe. Each possibility represents a separate embodiment of this invention.

Some pharmaceutical compositions have chemical stability problems, caused either by interaction of the active agent with the carrier and/or other excipient and/or by interaction of the active agent with, for example, the subject's skin—in case of topical compositions.

One of the solutions for this chemical stability problem is the encapsulation of ruxolitinib or pharmaceutically acceptable salt thereof (or deuterated ruxolitinib or pharmaceutically acceptable salt thereof) in the composition. The preferred encapsulation method of this invention is detailed in U.S. Pat. No. 9,687,465 and published U.S. Patent Application No. 2018147165 (to Sol-Gel Technologies), whose contents are enclosed herein in their entirety. Thus, for example, ruxolitinib or pharmaceutically acceptable salt thereof (or deuterated ruxolitinib or pharmaceutically acceptable salt thereof) in the compositions of this invention may be encapsulated as disclosed above.

In another embodiment, the composition is encapsulated according to the process described in Example 2 herein below.

In another embodiment, this invention provides a gel comprising encapsulated ruxolitinib phosphate, as described in Example 3.

In some embodiments, the ruxolitinib or pharmaceutically acceptable salt thereof, or deuterated ruxolitinib or a pharmaceutically acceptable salt thereof of the composition provided herein is micronized.

Pharmaceutical carriers or vehicles suitable for preparation of the compositions provided herein include any such carriers known to those skilled in the art to be suitable for the particular mode of administration.

Also provided herein is a topical combination composition comprising ruxolitinib, deuterated ruxolitinib or pharmaceutically acceptable salt and at least one additional active agent selected from about 0.01% w/w to about 0.25% w/w a low-dose steroid, from about 0.25% w/w to about 3.0% w/w tapinarof, from about 0.25% w/w to about 3% w/w of a PDE4 inhibitor; from about 0.25% w/w to about 3% w/w a AhR agonist and a carrier suitable for topical administration.

In another embodiment, provided herein is a topical combination composition comprising ruxolitinib, deuterated ruxolitinib or pharmaceutically acceptable salt and at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and a carrier suitable for topical administration.

In another embodiment, provided herein is a topical combination composition comprising ruxolitinib, deuterated ruxolitinib or pharmaceutically acceptable salt and from about 0.25% w/w to about 3.0% w/w tapinarof and a carrier suitable for topical administration.

In another embodiment, provided herein is a topical combination composition comprising ruxolitinib, deuterated ruxolitinib or pharmaceutically acceptable salt and at least one additional active agent selected from about 0.01% w/w to about 0.25% w/w a low-dose steroid, and a carrier suitable for topical administration.

In another embodiment, provided herein is a topical combination composition comprising ruxolitinib, deuterated ruxolitinib or pharmaceutically acceptable salt and from about 0.25% w/w to about 3% w/w of roflumilast and a carrier suitable for topical administration.

Low-dose steroids include corticosteroid or glucocorticoids including cortisone, hydrocortisone and prednisone, clobetasol propionate, flurandrenolide, betamethasone dipropionate, diflorasone diacetate, desoxymethasone, fluocinonide, betamethasone dipropionate, mometasone furoate, diflorasone diacetate, halcinonide, fluocinonide, desoxymethasone, fluticasone propionate, fluocinonide, betamethasone valerate, flurandrenolide, mometasone furoate, triamcinolone acetonide, fluocinolone acetonide, desoxymethasone or hydrocortisone valerate.

The PDE4 inhibitor in the composition of this disclosure is selected from roflumilast, apremilast, crisaborole, rolipram, cilomilast, ibudilast, piclamilast and combinations thereof.

AhR agonist in the composition of this disclosure is selected from an imidazole active agent (ketoconazole, clotrimazole, econazole, tioconazole, miconazole and butoconazole) a triazole active agent (itraconazole, fluconazole, terconazole, voriconazole, eficonazole, albaconazole, ravuconazole and isavuconazole), a flavonoid (pentahydroxyflavonoids, hexahydroxyflavonoids, tetrahydroxyflavonoids, trihydroxyflavonoids, quercetin, apigenin) and combinations thereof.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, sebum control products or any other suitable formulation. The preferred compositions are the cream, the lotion, the gel and the foam. Each possibility represents a separate embodiment of this invention.

A drug penetrates the skin into the vasculature then undergoes distribution into the tissues, metabolism and excretion characteristic of the drug further delaying detection in the blood and extending lag time. The lag time measured using in vitro permeation testing (IVPT) is shorter than the lag time measured in PK experiments, because achieving measurable blood levels of active always takes longer than for active to diffuse to the depth in the skin required to reach the vasculature for outflux from the skin. The lag time depends on the compound penetrating the skin and may be an hour or more. Skin penetration enhancers, excipients combined with the pharmaceutical active to formulate a topical product, can influence lag time as well as increase the amount of active crossing the stratum corneum.

In some embodiments, the compositions described herein have decreased skin permeability. In other embodiments, the topical composition described herein has a low systemic absorption. In other embodiments, the topical composition described herein has no systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the composition has minor or negligible permeation capacity through the skin within the first hour of administration.

In another embodiments, the composition has minor permeation capacity through the skin within the first hour of administration. In another embodiments, the composition has negligible permeation capacity through the skin within the first hour of administration. In another embodiments, the composition has minor or negligible permeation capacity through the skin within the first hour of administration.

In some embodiments, the compositions described herein have decreased skin permeability compared to, for example, OPZELURA (Incyte Corporation). In other embodiments, the topical composition described herein has a low systemic absorption compared to, for example, OPZELURA (Incyte Corporation). In other embodiments, the topical composition described herein has no systemic absorption. In other embodiments, the composition has a skin penetration lag time longer than one hour as measured by in vitro permeation testing (IVPT) and longer compared to, for example, OPZELURA (Incyte Corporation). In other embodiments, the composition has a skin penetration lag time is between 2 hours to 24 hours. In other embodiments, the composition has minor or negligible permeation capacity through the skin within the first hour of administration compared to, for example, OPZELURA (Incyte Corporation).

The present invention provides a topical composition comprising more than 1.5% w/w ruxolitinib or pharmaceutically acceptable salt thereof, or deuterated ruxolitinib or a pharmaceutically acceptable salt thereof and a pharmaceutically acceptable carrier. In one embodiment, the ruxolitinib or pharmaceutically acceptable salt thereof, or deuterated ruxolitinib or a pharmaceutically acceptable salt thereof is in an amount of more than 1.5% to about 5.0% w/w. In another embodiment, the composition has decreased skin permeability. In other embodiments, the topical composition has a low systemic absorption. In other embodiments, the topical composition has no systemic absorption. In other embodiments, the composition has a skin penetration lag time longer than one hour as measured by in vitro permeation testing (IVPT). In another embodiments, the composition has negligible permeation capacity through the skin within the first hour of administration.

In one embodiment, provided herein a topical composition comprising from about more than 1.5% w/w ruxolitinib or in an amount of more than 1.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the composition is formulated as a gel. In certain embodiments, the composition is formulated as a gel, wherein the ruxolitinib is dispersed or fully or partially dissolved therein.

In one embodiment, provided herein a topical composition comprising from about more than 1.5% w/w ruxolitinib or in an amount of more than 1.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the ruxolitinib or pharmaceutically acceptable salt thereof, or deuterated ruxolitinib or a pharmaceutically acceptable salt thereof is not dissolved or partially dissolved in the composition.

In one embodiment, provided herein a topical composition comprising from about more than 1.5% w/w ruxolitinib or in an amount of more than 1.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the ruxolitinib is fully or partially dissolved by a solvent, and thereby possessing less permeation capacity than formulations disclosed in the prior art such as, for example, OPZELURA (Incyte Corporation)

In one embodiment, provided herein a topical composition comprising from about more than 0.5% w/w ruxolitinib or in an amount of more than 0.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the composition is formulated as a gel. In certain embodiments, the composition is formulated as a gel, wherein the ruxolitinib is dispersed or fully or partially dissolved therein, and wherein the composition has decreased skin permeability, no systemic absorption or low systemic absorption.

In one embodiment, provided herein a topical composition comprising from about more than 0.5% w/w ruxolitinib or in an amount of more than 0.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the ruxolitinib or pharmaceutically acceptable salt thereof, or deuterated ruxolitinib or a pharmaceutically acceptable salt thereof is micronized.

In one embodiment, provided herein a topical composition comprising from about more than 0.5% w/w ruxolitinib or in an amount of more than 0.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the ruxolitinib or pharmaceutically acceptable salt thereof, or deuterated ruxolitinib or a pharmaceutically acceptable salt thereof is not dissolved or partially dissolved in the composition, and wherein the composition has decreased skin permeability, no skin absorption or low systemic absorption.

In one embodiment, provided herein a topical composition comprising from about more than 0.5% w/w ruxolitinib or in an amount of more than 0.5% to about 5.0% w/w ruxolitinib or pharmaceutically acceptable salt thereof, or deuterated ruxolitinib or a pharmaceutically acceptable salt thereof; and a pharmaceutically acceptable carrier, wherein the ruxolitinib is fully or partially dissolved by a solvent, and thereby possessing less permeation capacity than formulations disclosed in the prior art, and wherein the composition has decreased skin permeability or low systemic absorption compared to, for example, OPZELURA (Incyte Corporation).

Sebum control products may include ingredients selected from the group consisting of: azelaic acid, salicylic acid, sulfur, nicotinamide, L-carnitine and combinations thereof. Each possibility represents a separate embodiment of this invention.

The active agent ruxolitinib or pharmaceutically acceptable salt thereof (or deuterated ruxolitinib or a pharmaceutically acceptable salt thereof) is included in the carrier in an amount sufficient to exert a therapeutically useful effect i.e., amelioration of the symptoms of an inflammatory skin conditions, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment. Each possibility represents a separate embodiment of this invention.

In some embodiment, the compositions of this invention comprise ruxolitinib or pharmaceutically acceptable salt thereof (or deuterated ruxolitinib or a pharmaceutically acceptable salt thereof); and a carrier, a solvent, an emollient, a surfactant, an anti-oxidant, a chelating agent, a gelling agent, a wetting agent, a penetration enhancer, a preservative (e.g. imidurea and methylparaben), an anti-oxidant, a buffer or any combination thereof. Each possibility represents a separate embodiment of this invention.

In some embodiments, the compositions of this invention comprise a penetration enhancer. In another embodiment, the penetration enhancer is selected from the group consisting of dimethylsulfoxide (DMSO), diethylene glycol monoethyl ether (Transcutol), methylsulfonylmethane (MSM), oleic acid, oleyl alcohol, propylene glycol, dimethyl isosorbide, isopropyl myristate, diethyl sebacate, ethanol, isopropyl alcohol, a polyethylene glycol, hexylene glycol, glycofurol and any combination thereof. Each possibility represents a separate embodiment of this invention.

In some embodiments, the compositions of this invention comprise an emollient. In another embodiment, the emollient is selected from the group consisting of castor oil, mineral oil, vegetable oil, soybean oil, shea butter, cocoa butter, paraffin, beeswax, oleic acid, squalene, cetyl alcohol, isopropyl myristate, urea, glycerol, propylene glycol, lactic acid and any combination thereof. Each possibility represents a separate embodiment of this invention.

In some embodiments, the compositions of this invention comprise a chelating agent. In another embodiment, the chelating agent is selected from the group consisting of EDTA deferoxamine, deferiprone, deferasirox, dimercaptosuccinic acid (succimer) triethylenetetramine (trientine) and any combination thereof. Each possibility represents a separate embodiment of this invention.

In some embodiments, the compositions of this invention comprise an anti-oxidant. In another embodiment, the anti-oxidant is selected from the group consisting of butylated hydroxytoluene (BHT), parabens, propyl gallate, ascorbic acid, flavones, flavanones, flavonols, stilbenoids, gallic acid, cinnamic acid, ellagic acid, salicylic acid, curcumin, eugenol, citric acid and any combination thereof. Each possibility represents a separate embodiment of this invention.

In some embodiments, the compositions of this invention comprise a solvent. In another embodiment the solvent is selected from the group consisting of propylene glycol, diethylene glycol monoethyl ether (Transcutol), dimethylsulfoxide (DMSO), diethyl sebacate, polyethylene glycol, oleyl alcohol, isosorbide dimethyl ether, ethanol, isopropyl alcohol, isopropyl myristate, oleic acid, hexylene glycol, glycerin, glycofurol and any combination thereof. Each possibility represents a separate embodiment of this invention.

In some embodiments, the compositions of this invention comprise a preservative. In another embodiment the preservative is selected from the group consisting of benzoic acid, methylparaben, imidurea, chlorocresol, Phenoxyethanol, propyl paraben, benzalkonium chloride, benzyl alcohol, imidazolidinyl urea, sodium benzoate, sorbic acid, thimerosal sorbic acid, sodium sorbate, sodium sulfite, ethanol, tocopherols and any combination thereof. In another embodiment, the preservative is imidurea and methylparaben. Each possibility represents a separate embodiment of this invention.

In some embodiments, the compositions of this invention comprise a buffer. In another embodiment, the buffer is selected from the group consisting of citric acid, phosphoric acid, acetic acid, tartaric acid, glutamic acid, aspartic acid, malic acid, succinic acid, fumaric acid, salt thereof, and any combination thereof. Each possibility represents a separate embodiment of this invention.

In some embodiments, the compositions of this invention comprise a gelling agent. In another embodiment, the gelling agent is selected from the group consisting of carbomer homopolymer type A (Carbopol® 981, Carbopol® 980), Sepineo P600, Natrosol hydroxyethylcellulose, starch, pectin, gelatin, agar, alginic acid and salts thereof, Carbomer® 934, carboxymethylcellulose, hydroxypropylmethylcellulose and any combination thereof. Each possibility represents a separate embodiment of this invention.

In some embodiments, the compositions of this invention comprise a surfactant. In another embodiment, the surfactant is selected from the group consisting of carbomer copolymer type B (Pemulen®TR-1), sorbitan monooleate (Span80), polysorbate 80 (Tween 80), polysorbate 20, polysorbate 60 (Tween 60), sodium dodecylsulfate, cetearyl alcohol, dioctyl sodium sulfosuccinate, glyceryl oleate, glyceryl stearate, poloxamers and any combination thereof. Each possibility represents a separate embodiment of this invention.

In some embodiments, the compositions of this invention comprise a wetting agent. In another embodiment, the wetting agent is selected from the group consisting of PEG 400, glycerin, poloxamers, sorbitan monooleate (Span80), polysorbate 80 (Tween 80), polysorbate 20, polysorbate 60 (Tween 60), benzalkonium chloride, sodium dodecylsulfate and any combination thereof. Each possibility represents a separate embodiment of this invention.

Suitable pharmaceutically and dermatologically acceptable vehicles for topical application include lotions, creams, foams, solutions, gels, patches and the like. Generally, the vehicle is either organic in nature or an aqueous emulsion and capable of accommodating the selected active agent(s), which may be micronized, dispersed, suspended or dissolved therein. Each possibility represents a separate embodiment of this invention. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents. Each possibility represents a separate embodiment of this invention.

### Regimen of Treatment

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier.

This invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of a PDE4 inhibitor; from about 0.25% w/w to about 3% w/w a AhR agonist and any combination thereof, and a pharmaceutically acceptable carrier.

This invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and any combination thereof, and a pharmaceutically acceptable carrier.

This invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast and any combination thereof, and a pharmaceutically acceptable carrier

This invention provides a regimen for the treatment, prevention or alleviation of an inflammatory skin condition comprising topically applying onto an affected skin area of a subject in need thereof, once a day, a topical composition which comprises more than 0.5% w/w to about 5.0% w/w ruxolitinib, deuterated ruxolitinib or a pharmaceutically acceptable salt thereof, wherein the composition has decreased skin permeability. In other embodiments, the topical composition has no systemic absorption. In other embodiments, the topical composition has a low systemic absorption. In other embodiments, the composition has a skin penetration lag time longer than one hour as measured by in vitro permeation testing (IVPT). In other embodiments, the composition has a skin penetration lag time between 2 hours to 24 hours. In another embodiment, the composition has minor or negligible permeation capacity through the skin within the first hour of administration.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or a pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

This invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of a PDE4 inhibitor; from about 0.25% w/w to about 3% w/w a AhR agonist and any combination thereof, and a pharmaceutically acceptable carrier, wherein the composition has decreased skin permeability or low systemic absorption.

This invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and any combination thereof, and a pharmaceutically acceptable carrier, wherein the composition has decreased skin permeability, no systemic absorption or low systemic absorption.

This invention provides a regimen for the treatment, prevention or alleviation of an inflammatory skin condition comprising topically applying onto an affected skin area of a subject in need thereof, once or twice or three time a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast and any combination thereof, and a pharmaceutically acceptable carrier, wherein the composition has decreased skin permeability, no systemic absorption or low systemic absorption.

This invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, wherein the composition has decreased skin permeability or low systemic absorption. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In another embodiment, the composition has minor or negligible permeation capacity through the skin within the first hour of administration.

In another embodiment, provided herein is a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and any combination thereof; and a pharmaceutically acceptable carrier; wherein the composition has decreased skin permeability, no systemic absorption or low systemic absorption.

In another embodiment, the low-dose steroid, PDE4 inhibitor, AhR agonist, tapinarof or roflumilast are formulated separately and are administered concomitantly or sequentially with the ruxolitinib composition.

In another embodiment, the low-dose steroid, PDE4 inhibitor, AhR agonist, tapinarof and roflumilast are formulated in combination with the ruxolitinib composition.

In another embodiment, the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition.

In another embodiment, the regimen provides a comparable or lower side effects compared to a systemic administration of a corresponding ruxolitinib, deuterated ruxolitinib, or pharmaceutically acceptable salt thereof composition.

In another embodiment, the regimen provides a comparable or lower side effects compared to a topical administration of a corresponding ruxolitinib, deuterated ruxolitinib, or pharmaceutically acceptable salt thereof composition known in the art, for example OPZELURA (Incyte Corporation).

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition. (in terms of AUC).

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to systemic administration or a topical administration (for example OPZELURA (Incyte Corporation) of a corresponding ruxolitinib, deuterated ruxolitinib, or pharmaceutically acceptable salt thereof composition. (in terms of AUC).

In some embodiments, this invention provides a regimen for the treatment, prevention or alleviation of an inflammatory skin condition comprising topically applying onto an affected skin area of a subject in need thereof, at least once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to a twice daily systemic or topical absorption of a corresponding ruxolitinib, deuterated ruxolitinib, or pharmaceutically acceptable salt thereof composition (in terms of AUC), wherein the composition has decreased skin permeability or low systemic absorption. In another embodiment, the composition is administered once daily, twice daily or three times per day.

In some embodiments, this invention provides a regimen for the treatment, prevention or alleviation of an inflammatory skin condition comprising topically applying onto an affected skin area of a subject in need thereof, at least once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to systemic administration or a topical administration of a corresponding ruxolitinib, deuterated ruxolitinib, or pharmaceutically acceptable salt thereof composition (in terms of AUC) known in the art, wherein the composition has decreased skin permeability or low systemic absorption. In another embodiment, the composition is administered once daily, twice daily or three times per day.

In some embodiments, the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In another aspect of this invention, the inflammatory skin condition is acne. In another aspect of this invention, the inflammatory skin condition is rosacea. In another aspect of this invention, the inflammatory skin condition is atopic dermatitis. In another aspect of this invention, the inflammatory skin condition is psoriasis. In another aspect of this invention, the inflammatory skin condition is flexural/inverse psoriasis. In another aspect of this invention, the inflammatory skin condition is eczema. In another aspect of this invention, the inflammatory skin condition is contact dermatitis. In another aspect of this invention, the inflammatory skin condition is urticaria. In another aspect of this invention, the inflammatory skin condition is dermatitis herpetiformis. In another aspect of this invention, the inflammatory skin condition is lichen planus. In another aspect of this invention, the inflammatory skin condition is seborrheic dermatitis.

In some embodiments, the effective amount is a therapeutically effective amount of the ruxolitinib or pharmaceutically acceptable salt thereof (or deuterated ruxolitinib or a pharmaceutically acceptable salt thereof), namely an amount which will cure, treat, prevent or alleviate an inflammatory skin condition, specifically atopic dermatitis.

Dosage frequencies can be gradually decreased over time and maintained at a steady dose suitable for long-term—six months, 1 year, 5 years, 10 years or more, up to lifelong administration to control the symptoms of the inflammatory skin condition, or specifically of atopic dermatitis. For example, dosage administration can begin at from once a day, to two times a week, to once a week, to once every two weeks or less frequent than once every two weeks. Each possibility represents a separate embodiment of this invention.

In some embodiments, the compositions of this invention are used in the methods as described hereinabove and/or are used in the preparation of medicaments that are used in said methods.

### Methods of Treatment

In one additional aspect, this invention provides a method of treatment, prevention or alleviation of an inflammatory skin condition comprising administering, to a subject in need thereof, once a day, a topical pharmaceutical composition or the dosage form as described herein.

In some aspects this invention provides a method of treatment, prevention or alleviation of an inflammatory skin condition, wherein the method comprises administering to a subject in need thereof, once a day, a topical composition a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier.

This invention provides a method for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of a PDE4 inhibitor; from about 0.25% w/w to about 3% w/w a AhR agonist and any combination thereof, and a pharmaceutically acceptable carrier.

This invention provides a method for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and any combination thereof; and a pharmaceutically acceptable carrier. This invention provides a method for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof, wherein the composition has decreased skin permeability. In other embodiments, the topical composition has a low systemic absorption. In other embodiments, the composition has a skin penetration lag time longer than one hour as measured by in vitro permeation testing (IVPT). In other embodiments, the composition has a skin penetration lag time is between 2 hours to 24 hours. In other embodiments, the composition has minor or negligible permeation capacity through the skin within the first hour of administration.

This invention provides a method for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib, deuterated ruxolitinib or a pharmaceutically acceptable salt thereof; in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast and any combination thereof; and a pharmaceutically acceptable carrier

In another embodiment, the low-dose steroid, PDE4 inhibitor, AhR agonist, tapinarof or roflumilast are formulated separately and are administered concomitantly or sequentially with the ruxolitinib composition.

In another embodiment, the low-dose steroid, PDE4 inhibitor, AhR agonist, tapinarof and roflumilast are formulated in combination with the ruxolitinib composition.

In some aspects this invention provides a method of treatment, prevention or alleviation of an inflammatory skin condition, wherein the method comprises administering to a subject in need thereof, once a day, a topical composition a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition. (in terms of AUC).

In some aspects this invention provides a method of treatment, prevention or alleviation of an inflammatory skin condition, wherein the method comprises administering to a subject in need thereof, once a day, a topical composition a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to systemic administration or a topical administration (for example OPZELURA (Incyte Corporation) of a corresponding ruxolitinib, deuterated ruxolitinib, or pharmaceutically acceptable salt thereof composition. (in terms of AUC).

In some aspects this invention provides a method of treatment, prevention or alleviation of an inflammatory skin condition, wherein the method comprises administering to a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier; and wherein the composition has decreased skin permeability, no systemic absorption or low systemic absorption.

This invention provides a method for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of a PDE4 inhibitor; from about 0.25% w/w to about 3% w/w a AhR agonist and any combination thereof, and a pharmaceutically acceptable carrier; and wherein the composition has decreased skin permeability, no systemic absorption or low systemic absorption.

This invention provides a method for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast; from about 0.25% w/w to about 3% w/w a AhR agonist and any combination thereof, and a pharmaceutically acceptable carrier, and wherein the composition has decreased skin permeability, no systemic absorption or low systemic absorption.

In other embodiments, the composition has a skin penetration lag time longer than one hour as measured by in vitro permeation testing (IVPT). In other embodiments, the composition has a skin penetration lag time is between 2 hours to 24 hours. In other embodiments, the composition has minor or negligible permeation capacity through the skin within the first hour of administration.

This invention provides a method for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib, deuterated ruxolitinib or a pharmaceutically acceptable salt thereof, in combination with at least one additional active agent selected from about 0.01% w/w to about 0.25% w/w a low-dose steroid, from about 0.25% w/w to about 3.0% w/w tapinarof, from about 0.25% w/w to about 3% w/w of roflumilast and any combination thereof; and a pharmaceutically acceptable carrier, and wherein the composition has decreased skin permeability, no systemic absorption or low systemic absorption.

In another embodiment, the low-dose steroid, PDE4 inhibitor, AhR agonist, tapinarof or roflumilast are formulated separately and are administered concomitantly or sequentially with the ruxolitinib composition.

In another embodiment, the low-dose steroid, PDE4 inhibitor, AhR agonist, tapinarof and roflumilast are formulated in combination with the ruxolitinib composition.

In some aspects this invention provides a method of treatment, prevention or alleviation of an inflammatory skin condition, wherein the method comprises administering to a subject in need thereof, at least once a daily, a topical composition a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption, and wherein the regimen provides a comparable or lower side effects compared to a twice daily systemic or topical absorption of a corresponding ruxolitinib, deuterated ruxolitinib, or pharmaceutically acceptable salt thereof composition (in terms of AUC). In another embodiment, the topical composition is administered once daily, twice daily or three times per day.

In some aspects this invention provides a method of treatment, prevention or alleviation of an inflammatory skin condition, wherein the method comprises administering to a subject in need thereof, at least once a daily, a topical composition a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption, and wherein the regimen provides a comparable or lower side effects compared to systemic administration or a topical administration (for example OPZELURA (Incyte Corporation) of a corresponding ruxolitinib, deuterated ruxolitinib, or pharmaceutically acceptable salt thereof composition. (in terms of AUC).

In another embodiment, the topical composition is administered once daily, twice daily or three times per day.

In some embodiments, the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In another aspect of this invention, the inflammatory skin condition is acne. In another aspect of this invention, the inflammatory skin condition is rosacea. In another aspect of this invention, the inflammatory skin condition is atopic dermatitis. In another aspect of this invention, the inflammatory skin condition is psoriasis. In another aspect of this invention, the inflammatory skin condition is flexural/inverse psoriasis. In another aspect of this invention, the inflammatory skin condition is eczema. In another aspect of this invention, the inflammatory skin condition is contact dermatitis. In another aspect of this invention, the inflammatory skin condition is urticaria. In another aspect of this invention, the inflammatory skin condition is dermatitis herpetiformis. In another aspect of this invention, the inflammatory skin condition is lichen planus. In another aspect of this invention, the inflammatory skin condition is seborrheic dermatitis.

In some embodiments, the effective amount is a therapeutically effective amount of the ruxolitinib or pharmaceutically acceptable salt thereof (or deuterated ruxolitinib or a pharmaceutically acceptable salt thereof), namely an amount which will cure, treat, prevent or alleviate an inflammatory skin condition, specifically atopic dermatitis.

Dosage frequencies can be gradually decreased over time and maintained at a steady dose suitable for long-term—six months, 1 year, 5 years, 10 years or more, up to lifelong administration to control the symptoms of the inflammatory skin condition, or specifically of atopic dermatitis. For example, dosage administration can begin at from once a day, to two times a week, to once a week, to once every two weeks or less frequent than once every two weeks. Each possibility represents a separate embodiment of this invention.

In some embodiments, the compositions of this invention are used in the methods as described hereinabove and/or are used in the preparation of medicaments that are used in said methods.

In one further aspect, this invention provides a kit comprising the compositions or dosage forms as described hereinabove. In one embodiment, the kit further comprises instructions for administration.

The compositions provided herein can be packaged as articles of manufacture containing packaging material, a composition provided herein, and a label that indicates that the composition is for treating an inflammatory skin disease, specifically atopic dermatitis, and is formulated for e.g. topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes or dual chamber application syringes and any packaging material suitable for the selected formulation and intended mode of administration and treatment. Each possibility represents a separate embodiment of this invention.

### EMBODIMENTS

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w or more than 1.5% w/w to about 3% w/w.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier; wherein the treatment comprises a combination treatment with at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof.

In some embodiments, this invention provides a regimen for the treatment, prevention or alleviation of inflammatory skin condition comprising topically applying onto an affected skin area of a subject in need thereof, once a day, a topical composition which comprises ruxolitinib phosphate in an amount of more than 1.5% w/w to about 5.0% w/w and a pharmaceutically acceptable carrier. In one embodiment, the ruxolitinib phosphate is in an amount of from about 2.0% w/w to about 2.5% w/w or between more than 1.5% w/w to about 3% w/w.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the ruxolitinib or pharmaceutically acceptable salt thereof, or deuterated ruxolitinib or a pharmaceutically acceptable salt thereof is not dissolved or partially dissolved in the composition.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the ruxolitinib or pharmaceutically acceptable salt thereof, or deuterated ruxolitinib or a pharmaceutically acceptable salt thereof is micronized.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability or low systemic absorption.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has low systemic absorption with a skin penetration lag time being between 2 hours to 24 hours.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has minor or negligible permeation capacity through the skin within the first hour of administration.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition (in terms of AUC).

In some embodiments, this invention provides a regimen for the treatment, prevention or alleviation of inflammatory skin condition comprising topically applying onto an affected skin area of a subject in need thereof, once a day, a topical composition which comprises ruxolitinib phosphate in an amount of more than 1.5% w/w to about 5.0% w/w and a pharmaceutically acceptable carrier, wherein the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition (in terms of AUC).

In other embodiments, the side effects of ruxolitinib or pharmaceutically acceptable salt thereof, or deuterated ruxolitinib or a pharmaceutically acceptable salt include anemia, balance impairment, dizziness, headache, labyrinthitis, meniere's disease, neutropenia, thrombocytopenia, vertigo, diarrhea, skin rash, black, tarry stools, bladder pain, bleeding gums, orthostatic dizziness, weight gain, flatulence or combination thereof. In other embodiments, the side effects of ruxolitinib or pharmaceutically acceptable salt thereof, or deuterated ruxolitinib or a pharmaceutically acceptable salt are as disclosed in https://www.drugs.com/sfx/ruxolitinib-side-effects.html.

In some embodiments, this invention provides a regimen for the treatment, prevention or alleviation of inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w or more than 1.5% w/w to about 3% w/w.

In some embodiments, this invention provides a regimen for the treatment, prevention or alleviation of inflammatory skin condition comprising topically applying onto an affected skin area of a subject in need thereof, once a day, a topical composition which comprises more than 1.5% w/w to about 5.0% w/w ruxolitinib phosphate and a pharmaceutically acceptable carrier, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w or more than 1.5% w/w to about 3% w/w.

In some embodiments, this invention provides a regimen for the treatment, prevention or alleviation of inflammatory skin condition comprising topically applying onto an affected skin area of a subject in need thereof, once a day, a topical composition which comprises more than 1.5% w/w to about 5.0% w/w ruxolitinib phosphate and a pharmaceutically acceptable carrier, wherein the composition is formulated as a gel or as a gel. In one embodiment, the ruxolitinib phosphate is in an amount of from about 2.0% w/w to about 2.5% w/w; between more than 1.5% w/w to about 3% w/w; or between 1.6% w/w to about 3% w/w.

In some embodiments, this invention provides a regimen for the treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or more than 1.5% w/w to about 3% w/w; or between 1.6% w/w to about 3% w/w. In another embodiment, the inflammatory skin condition is atopic dermatitis.

In some embodiments, this invention provides a regimen for the treatment, prevention or alleviation of inflammatory skin condition comprising topically applying onto an affected skin area of a subject in need thereof, once a day, a topical composition which comprises ruxolitinib phosphate in an amount of more than 1.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib phosphate is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, this invention provides a regimen for the treatment, prevention or alleviation of inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or more than 1.5% w/w to about 3% w/w; or between 1.6% w/w to about 3% w/w. In another embodiment, the inflammatory skin condition is atopic dermatitis.

This invention further provides a method of treatment, prevention or alleviation of an inflammatory skin condition, wherein the method comprises administering to a subject in need thereof, once a day, a topical composition a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w or between more than 1.5% w/w to about 3% w/w. In another embodiment, the inflammatory skin condition is atopic dermatitis.

This invention further provides a method of treatment, prevention or alleviation of an inflammatory skin condition, wherein the method comprises administering to a subject in need thereof, once a day, a topical composition a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier; wherein the method comprises further administering at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof and a pharmaceutically acceptable carrier.

In some embodiments, this invention provides a method of treatment, prevention or alleviation of an inflammatory skin condition, wherein the method comprises administering to a subject in need thereof, once a day, a topical pharmaceutical composition comprising ruxolitinib phosphate in an amount of more than 1.5% w/w to about 5.0% w/w and a pharmaceutically acceptable carrier. In one embodiment, the ruxolitinib phosphate is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, this invention provides a method of treatment, prevention or alleviation of an inflammatory skin condition comprising topically applying onto an affected skin area of a subject in need thereof, once a day, a topical composition which comprises more than 1.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the method provides a comparable or lower side effects compared to a twice daily of systemic administration; or comparable or lower side effects compared to twice daily of topical administration of a corresponding ruxolitinib, deuterated ruxolitinib, or pharmaceutically acceptable salt thereof composition (in terms of AUC).

In some embodiments, this invention provides a method of treatment, prevention or alleviation of inflammatory skin condition comprising topically applying onto an affected skin area of a subject in need thereof, once a day, a topical composition which comprises ruxolitinib phosphate in an amount of more than 1.5% w/w to about 5.0% w/w and a pharmaceutically acceptable carrier, wherein the method provides a comparable or lower side effects compared to a twice daily of systemic administration; or comparable or lower side effects compared to twice daily of topical administration of a corresponding ruxolitinib, deuterated ruxolitinib, or pharmaceutically acceptable salt thereof composition.

In other embodiments, the side effects of ruxolitinib or pharmaceutically acceptable salt thereof, or deuterated ruxolitinib or a pharmaceutically acceptable salt include anemia, balance impairment, dizziness, headache, labyrinthitis, meniere's disease, neutropenia, thrombocytopenia, vertigo, and orthostatic dizziness, weight gain, flatulence or combination thereof.

In some embodiments, this invention provides a method of treatment, prevention or alleviation of an inflammatory skin condition comprising topically applying onto an affected skin area of a subject in need thereof, once a day, a topical composition which comprises more than 1.5% w/w to about 3.0% w/w ruxolitinib or pharmaceutically acceptable salt thereof, or deuterated ruxolitinib or a pharmaceutically acceptable salt thereof and a pharmaceutically acceptable carrier. In another embodiment, the topical composition comprises more than 1.5% w/w to about 3.0% w/w ruxolitinib phosphate and a pharmaceutically acceptable carrier.

In some embodiments, this invention provides a method of treatment, prevention or alleviation of an inflammatory skin condition comprising topically applying onto an affected skin area of a subject in need thereof, once a day, a topical composition which comprises more than 2% w/w to about 2.5% w/w ruxolitinib or pharmaceutically acceptable salt thereof, or deuterated ruxolitinib or a pharmaceutically acceptable salt thereof and a pharmaceutically acceptable carrier. In another embodiment, the topical composition comprises about 2.0% w/w to about 2.5% w/w ruxolitinib phosphate and a pharmaceutically acceptable carrier.

In some embodiments, this invention provides a method of treatment, prevention or alleviation of an inflammatory skin condition comprising topically applying onto an affected skin area of a subject in need thereof, once a day, a topical composition which comprises about 2.5% w/w ruxolitinib or pharmaceutically acceptable salt thereof, or deuterated ruxolitinib or a pharmaceutically acceptable salt thereof and a pharmaceutically acceptable carrier. In another embodiment, the topical composition comprises about 2.5% w/w ruxolitinib phosphate and a pharmaceutically acceptable carrier.

In some embodiments, this invention provides a method of treatment, prevention or alleviation of an inflammatory skin condition, wherein the method comprises administering to a subject in need thereof, once a day, a topical pharmaceutical composition comprising ruxolitinib or pharmaceutically acceptable salt thereof, or deuterated ruxolitinib or a pharmaceutically acceptable salt thereof in an amount of more than 1.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once a day, a topical composition comprising ruxolitinib phosphate in an amount of more than 1.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib phosphate is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once a day, a topical pharmaceutical composition comprising ruxolitinib or pharmaceutically acceptable salt thereof, or deuterated ruxolitinib or a pharmaceutically acceptable salt thereof in an amount of more than 1.5% w/w to about 5.0% w/w and a pharmaceutically acceptable carrier, wherein the inflammatory skin condition is atopic dermatitis. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or between more than 1.5% w/w to about 3% w/w; or between 1.6% w/w to about 3% w/w.

In some embodiments, this invention provides a method of treatment, prevention or alleviation of an inflammatory skin condition, wherein the method comprises administering to a subject in need thereof, once a day, a topical composition comprising ruxolitinib phosphate in an amount of more than 1.5% w/w to about 5.0% w/w and a pharmaceutically acceptable carrier, wherein the inflammatory skin condition is atopic dermatitis. In one embodiment, the ruxolitinib phosphate is in an amount of from about 2.0% w/w to about 2.5% w/w; or between more than 1.5% w/w to about 3% w/w; or between 1.6% w/w to about 3% w/w.

In some embodiments, this invention provides a method of treatment, prevention or alleviation of an inflammatory skin condition, wherein the method comprises administering to a subject in need thereof, once a day, a topical pharmaceutical composition comprising ruxolitinib or pharmaceutically acceptable salt thereof, or deuterated ruxolitinib or a pharmaceutically acceptable salt thereof in an amount of more than 1.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis; and wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once a day, a topical composition comprising ruxolitinib phosphate in an amount of more than 1.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis; and wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib phosphate is in an amount of from about 2.0% w/w to about 2.5% w/w; or between more than 1.5% w/w to about 3% w/w; or between 1.6% w/w to about 3% w/w. In another embodiment, the inflammatory skin condition is atopic dermatitis.

In some embodiments, the ruxolitinib or pharmaceutically acceptable salt thereof, or deuterated ruxolitinib or a pharmaceutically acceptable salt thereof (e.g. phosphate salt) of the compositions or dosage forms of this invention—is encapsulated.

In some embodiments, the ruxolitinib or pharmaceutically acceptable salt thereof, or deuterated ruxolitinib or a pharmaceutically acceptable salt thereof (e.g. phosphate salt) of the compositions or dosage forms of this invention—is micronized.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

In one embodiment, the ruxolitinib or pharmaceutically acceptable salt thereof, or deuterated ruxolitinib or a pharmaceutically acceptable salt thereof is in an amount of from about 2.0% w/w to about 2.5% w/w; or more than 1.5% w/w to about 3% w/w; or between 1.6% w/w to about 3% w/w.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier; wherein the composition has decreased skin permeability or low systemic absorption, and wherein the treatment comprises a combination treatment with at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof.

In some embodiments, this invention provides a regimen for the treatment, prevention or alleviation of inflammatory skin condition comprising topically applying onto an affected skin area of a subject in need thereof, once or twice or three times a day, a topical composition which comprises ruxolitinib phosphate in an amount of more than 0.5% w/w to about 5.0% w/w and a pharmaceutically acceptable carrier, wherein the composition has decreased skin permeability, no systemic absorption or low systemic absorption. In one embodiment, the ruxolitinib phosphate is in an amount of from about 0.5% w/w to about 1.5% w/w; or between more than 0.5% w/w to about 3% w/w; or between 1.6% w/w to about 3% w/w.

In some embodiments, this invention provides a regimen for the treatment, prevention or alleviation of an inflammatory skin condition comprising topically applying onto an affected skin area of a subject in need thereof, once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the ruxolitinib or pharmaceutically acceptable salt thereof, or deuterated ruxolitinib or a pharmaceutically acceptable salt thereof is not dissolved or partially dissolved in the composition, and wherein the composition has decreased skin permeability, no systemic absorption or low systemic absorption.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the ruxolitinib or pharmaceutically acceptable salt thereof, or deuterated ruxolitinib or a pharmaceutically acceptable salt thereof is micronized, and wherein the composition has decreased skin permeability, no systemic absorption or low systemic absorption.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has low systemic absorption with a skin penetration lag time being between 2 hours to 24 hours.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has minor or negligible permeation capacity through the skin within the first hour of administration, and wherein the composition has decreased skin permeability, no systemic absorption or low systemic absorption.

In some embodiments, this invention provides a regimen for the treatment, prevention or alleviation of an inflammatory skin condition comprising topically applying onto an affected skin area of a subject in need thereof, once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the regimen provides a comparable or lower side effects compared to a twice daily systemic or topical absorption of a corresponding ruxolitinib, deuterated ruxolitinib, or pharmaceutically acceptable salt thereof composition (in terms of AUC), and wherein the composition has decreased skin permeability, no systemic absorption or low systemic absorption.

In some embodiments, this invention provides a regimen for the treatment, prevention or alleviation of inflammatory skin condition comprising topically applying onto an affected skin area of a subject in need thereof, once a day, a topical composition which comprises ruxolitinib phosphate in an amount of more than 0.5% w/w to about 5.0% w/w and a pharmaceutically acceptable carrier, wherein the regimen provides a comparable or lower side effects compared to a twice daily systemic or topical absorption of a corresponding ruxolitinib, deuterated ruxolitinib, or pharmaceutically acceptable salt thereof composition (in terms of AUC), and wherein the composition has decreased skin permeability, no systemic absorption or low systemic absorption.

In other embodiments, the side effects of ruxolitinib or pharmaceutically acceptable salt thereof, or deuterated ruxolitinib or a pharmaceutically acceptable salt include anemia, balance impairment, dizziness, headache, labyrinthitis, meniere's disease, neutropenia, thrombocytopenia, vertigo, diarrhea, skin rash, black, tarry stools, bladder pain, bleeding gums, orthostatic dizziness, weight gain, flatulence or combination thereof. In other embodiments, the side effects of ruxolitinib or pharmaceutically acceptable salt thereof, or deuterated ruxolitinib or a pharmaceutically acceptable salt are as disclosed in https://www.drugs.com/sfx/ruxolitinib-side-effects.html.

In some embodiments, this invention provides a regimen for the treatment, prevention or alleviation of inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w or more than 0.5% w/w to about 3% w/w; or between 0.6% w/w to about 3% w/w, and wherein the composition has decreased skin permeability, no systemic absorption or low systemic absorption.

In some embodiments, this invention provides a regimen for the treatment, prevention or alleviation of inflammatory skin condition comprising topically applying onto an affected skin area of a subject in need thereof, once or twice or three times a day, a topical composition which comprises more than 0.5% w/w to about 5.0% w/w ruxolitinib phosphate and a pharmaceutically acceptable carrier, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or more than 0.5% w/w to about 3% w/w; or between 0.6% w/w to about 3% w/w, and wherein the composition has decreased skin permeability, no systemic absorption or low systemic absorption.

In some embodiments, this invention provides a regimen for the treatment, prevention or alleviation of inflammatory skin condition comprising topically applying onto an affected skin area of a subject in need thereof, once or twice or three times a day, a topical composition which comprises more than 0.5% w/w to about 5.0% w/w ruxolitinib phosphate and a pharmaceutically acceptable carrier, wherein the composition is formulated as a gel or as a gel. In one embodiment, the ruxolitinib phosphate is in an amount of from about 0.5% w/w to about 1.5% w/w; or between more than 0.5% w/w to about 3% w/w; or between 0.6% w/w to about 3% w/w, and wherein the composition has decreased skin permeability, no systemic absorption or low systemic absorption.

In some embodiments, this invention provides a regimen for the treatment, prevention or alleviation of an inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability or low systemic absorption, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or more than 0.5% w/w to about 3% w/w; or between 0.6% w/w to about 3% w/w. In another embodiment, the inflammatory skin condition is atopic dermatitis.

In some embodiments, this invention provides a regimen for the treatment, prevention or alleviation of inflammatory skin condition comprising topically applying onto an affected skin area of a subject in need thereof, once or twice or three times a day, a topical composition which comprises ruxolitinib phosphate in an amount of more than 0.5% w/w to about 5.0% w/w and a pharmaceutically acceptable carrier, wherein the composition has decreased skin permeability, no systemic absorption or low systemic absorption, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib phosphate is in an amount of from about 0.5% w/w to about 1.5% w/w; or between more than 0.5% w/w to about 3% w/w; or between 0.6% w/w to about 3% w/w. In another embodiment, the inflammatory skin condition is atopic dermatitis, and wherein the composition has decreased skin permeability, no systemic absorption or low systemic absorption.

In some embodiments, this invention provides a regimen for the treatment, prevention or alleviation of inflammatory skin condition comprising topically applying onto an affected skin area of a subject in need thereof, once or twice or three times a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability or low systemic absorption, wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or more than 0.5% w/w to about 3% w/w; or between 0.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once or twice or three times a day, a topical composition a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

This invention further provides a method of treatment, prevention or alleviation of an inflammatory skin condition, wherein the method comprises administering to a subject in need thereof, once or twice or three times a day, a topical composition a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w or between more than 0.5% w/w to about 3% w/w, wherein the composition has decreased skin permeability, no systemic absorption or low systemic absorption. In another embodiment, the inflammatory skin condition is atopic dermatitis.

This invention further provides a method of treatment, prevention or alleviation of an inflammatory skin condition, wherein the method comprises administering to a subject in need thereof, once or twice or three times or a day, a topical composition a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier; wherein the method comprises further administering at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof and a pharmaceutically acceptable carrier, wherein the composition has decreased skin permeability, no systemic absorption or low systemic absorption.

In some embodiments, this invention provides a method of treatment, prevention or alleviation of an inflammatory skin condition, wherein the method comprises administering to a subject in need thereof, once or twice or three times a day, a topical pharmaceutical composition comprising ruxolitinib phosphate in an amount of more than 0.5% w/w to about 5.0% w/w and a pharmaceutically acceptable carrier. In one embodiment, the ruxolitinib phosphate is in an amount of from about 0.5% w/w to about 1.5% w/w; or between more than 0.5% w/w to about 3% w/w; or between 0.6% w/w to about 3% w/w, wherein the composition has decreased skin permeability, no systemic absorption or low systemic absorption. In another embodiment, the inflammatory skin condition is atopic dermatitis.

In some embodiments, this invention provides a method of treatment, prevention or alleviation of an inflammatory skin condition comprising topically applying onto an affected skin area of a subject in need thereof, once a day, a topical composition which comprises more than 0.5% w/w to about 5.0% w/w ruxolitinib or pharmaceutically acceptable salt thereof, or deuterated ruxolitinib or a pharmaceutically acceptable salt thereof and a pharmaceutically acceptable carrier, wherein the method provides a comparable or lower side effects compared to a twice daily systemic or topical absorption of a corresponding ruxolitinib, deuterated ruxolitinib, or pharmaceutically acceptable salt thereof composition (in terms of AUC).

In some embodiments, this invention provides a method of treatment, prevention or alleviation of inflammatory skin condition comprising topically applying onto an affected skin area of a subject in need thereof, once a day, a topical composition which comprises ruxolitinib phosphate in an amount of more than 0.5% w/w to about 5.0% w/w and a pharmaceutically acceptable carrier, wherein the method provides a comparable or lower side effects compared to a twice daily systemic or topical absorption of a corresponding ruxolitinib, deuterated ruxolitinib, or pharmaceutically acceptable salt thereof composition.

In other embodiments, the side effects of ruxolitinib or pharmaceutically acceptable salt thereof, or deuterated ruxolitinib or a pharmaceutically acceptable salt include anemia, balance impairment, dizziness, headache, labyrinthitis, meniere's disease, neutropenia, thrombocytopenia, vertigo, and orthostatic dizziness, weight gain, flatulence or combination thereof.

In some embodiments, this invention provides a method of treatment, prevention or alleviation of an inflammatory skin condition comprising topically applying onto an affected skin area of a subject in need thereof, once or twice or three times a day, a topical composition which comprises more than 0.5% w/w to about 3.0% w/w ruxolitinib or pharmaceutically acceptable salt thereof, or deuterated ruxolitinib or a pharmaceutically acceptable salt thereof and a pharmaceutically acceptable carrier, and wherein the composition has decreased skin permeability, no systemic absorption or low systemic absorption. In another embodiment, the topical composition comprises more than 0.5% w/w to about 3.0% w/w ruxolitinib phosphate and a pharmaceutically acceptable carrier.

In some embodiments, this invention provides a method of treatment, prevention or alleviation of an inflammatory skin condition comprising topically applying onto an affected skin area of a subject in need thereof, once a day, a topical composition which comprises more than 0.5% w/w to about 1.5% w/w ruxolitinib or pharmaceutically acceptable salt thereof, or deuterated ruxolitinib or a pharmaceutically acceptable salt thereof and a pharmaceutically acceptable carrier, and wherein the composition has decreased skin permeability, no systemic absorption or low systemic absorption. In another embodiment, the topical composition comprises about 0.5% w/w to about 1.5% w/w ruxolitinib phosphate and a pharmaceutically acceptable carrier.

In some embodiments, this invention provides a method of treatment, prevention or alleviation of an inflammatory skin condition, wherein the method comprises administering to a subject in need thereof, once or twice or three times a day, a topical pharmaceutical composition comprising ruxolitinib or pharmaceutically acceptable salt thereof, or deuterated ruxolitinib or a pharmaceutically acceptable salt thereof in an amount of more than 0.5% w/w to about 5.0% w/w and a pharmaceutically acceptable carrier, wherein the composition has decreased skin permeability, no systemic absorption or low systemic absorption, and wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or between more than 0.5% w/w to about 3% w/w; or between 0.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once or twice or three times a day, a topical composition comprising ruxolitinib phosphate in an amount of more than 0.5% w/w to about 5.0% w/w and a pharmaceutically acceptable carrier, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis, and wherein the composition has decreased skin permeability, no systemic absorption or low systemic absorption. In one embodiment, the ruxolitinib phosphate is in an amount of from about 0.5% w/w to about 1.5% w/w; or between more than 0.5% w/w to about 3% w/w; or between 0.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once or twice or three times a day, a topical pharmaceutical composition comprising ruxolitinib or pharmaceutically acceptable salt thereof, or deuterated ruxolitinib or a pharmaceutically acceptable salt thereof in an amount of more than 0.5% w/w to about 5.0% w/w and a pharmaceutically acceptable carrier, wherein the inflammatory skin condition is atopic dermatitis, wherein the composition has decreased skin permeability, no systemic absorption or low systemic absorption. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or between more than 0.5% w/w to about 3% w/w; or between 0.6% w/w to about 3% w/w.

In some embodiments, this invention provides a method of treatment, prevention or alleviation of an inflammatory skin condition, wherein the method comprises administering to a subject in need thereof, once or twice or three times a day, a topical composition comprising ruxolitinib phosphate in an amount of more than 0.5% w/w to about 5.0% w/w and a pharmaceutically acceptable carrier, wherein the composition has decreased skin permeability, no systemic absorption or low systemic absorption, and wherein the inflammatory skin condition is atopic dermatitis. In one embodiment, the ruxolitinib phosphate is in an amount of from about 0.5% w/w to about 1.5% w/w; or between more than 0.5% w/w to about 3% w/w; or between 0.6% w/w to about 3% w/w.

In some embodiments, this invention provides a method of treatment, prevention or alleviation of an inflammatory skin condition, wherein the method comprises administering to a subject in need thereof, once or twice or three times a day, a topical pharmaceutical composition comprising ruxolitinib or pharmaceutically acceptable salt thereof, or deuterated ruxolitinib or a pharmaceutically acceptable salt thereof in an amount of more than 0.5% w/w to about 5.0% w/w and a pharmaceutically acceptable carrier, wherein the composition has decreased skin permeability, no systemic absorption or low systemic absorption, and wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis; and wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib or pharmaceutically acceptable salt thereof, or deuterated ruxolitinib or a pharmaceutically acceptable salt thereof is in an amount of from about 0.5% w/w to about 1.5% w/w; or between more than 0.5% w/w to about 3% w/w; or between 0.6% w/w to about 3% w/w. In another embodiment, the inflammatory skin condition is atopic dermatitis.

In some embodiments, this invention provides a method of treatment, prevention or alleviation of an inflammatory skin condition, wherein the method comprises administering to a subject in need thereof, once or twice or three times a day, a topical composition comprising ruxolitinib phosphate in an amount of more than 0.5% w/w to about 5.0% w/w and a pharmaceutically acceptable carrier, wherein the composition has decreased skin permeability, no systemic absorption or low systemic absorption, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis; and wherein the composition is formulated as a cream, a gel, an ointment, an emulsion, a solution, a suspension, an elixir, a lotion, a tincture, a paste, a foam, an aerosol, a spray, a patch, a transdermal patch or as an applicator syringe. In one embodiment, the ruxolitinib phosphate is in an amount of from about 0.5% w/w to about 1.5% w/w; or between more than 0.5% w/w to about 3% w/w; or between 0.6% w/w to about 3% w/w. In another embodiment, the inflammatory skin condition is atopic dermatitis.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

In some embodiment, this invention provides a composition comprising deuterated ruxolitinib. Ruxolitinib has the following structure:

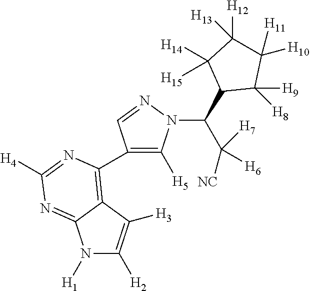

Deuterated ruxolitinib refers to the ruxolitinib compound wherein each of H1-H15 is independently deuterated. In another embodiment, H1-H15 are deuterated. In another embodiment, H8-H15 are deuterated. In another embodiment, H1-H4 are deuterated. In another embodiment, H5 is deuterated. In another embodiment, H6-H7 are deuterated. In another embodiment, H6, H7, H8, H9, H14 and H15 are deuterated.

As used herein, the indefinite articles “a” and “an” mean “at least one” or “one or more” unless the context clearly dictates otherwise.

As used herein, the term “treating” or“treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “agent” or “active agent” or “active pharmaceutical ingredient” or “API” are interchangeable and mean the ingredient is a pharmaceutical active agent which is biological active and is regulatory approved or approvable as such.

As used herein, the terms “inflammatory skin conditions”, “inflammatory skin disorders”, “inflammatory skin diseases” and “inflammatory cutaneous conditions” are any medical conditions affecting the integumentary system and are used interchangeably.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein “skin permeability” and “permeation capacity” are used herein interchangeably, and refer to the capacity or permeability of ruxolitinib, deuterated ruxolitinib or a pharmaceutically acceptable salt thereof to penetrate and diffuse through the skin through skin's outermost layer, the stratum corneum and reach the vasculature (blood system). In some embodiments, the compositions of this invention have decreased/low skin permeability compared to, for example, OPZELURA (Incyte Corporation).

As used herein “systemic absorption” is a route of administration of medication into the circulatory system so that the entire body is affected. Administration can take place via enteral administration (absorption of the drug through the gastrointestinal tract) or parenteral administration (generally injection, infusion, or implantation), as opposed to topical administration, where the effect is generally local. In another embodiment, the compositions described herein have no systemic absorption or low systemic absorption. In some embodiment, the bioavailablity/systemic absorption of the compositions described herein is calculated by the percentage of the amount of ruxolitinib that is quantifiable in plasma against the total amount of ruxolitinib in the ruxolitinib topical composition that was applied onto the skin lesions.

In another embodiment, there is no systemic absorption of the compositions described herein. In another embodiment, the systemic absorption refers to a median bioavailability of ruxolitinib, deuterated ruxolitinib or a pharmaceutically acceptable salt thereof within the compositions described herein. In another embodiment, the median bioavailability (systemic absorption) of the composition disclosed herein is less than 4.88 nM of ruxolitinib (for 0.5% of ruxolitinib, once daily administration). In another embodiment, the median bioavailability (systemic absorption) of the composition disclosed herein is less than 4.56 nM of ruxolitinib (for 1.5% of ruxolitinib, once daily administration). In another embodiment, the median bioavailability (systemic absorption) of the composition disclosed herein is less than 4.17 nM of ruxolitinib (for 1.5% of ruxolitinib, twice daily administration). In another embodiment, the median bioavailability (systemic absorption) of the composition disclosed herein is less than 4.75 nM of ruxolitinib (for 0.75% of ruxolitinib, twice daily administration). In another embodiment, the median bioavailability (systemic absorption) of the composition disclosed herein is less than 3.64 nM of ruxolitinib (for 1.5% of ruxolitinib, twice daily administration).

In another embodiment, the median bioavailability (systemic absorption) of the composition disclosed herein is between 0.01 nM to 5 nM of ruxolitinib (for 0.5% of ruxolitinib, once daily administration).

In another embodiment, the median bioavailability (systemic absorption) of the composition disclosed herein is between 0.01 nM to 5 nM of ruxolitinib (for 1.5% of ruxolitinib, once daily administration).

In another embodiment, the median bioavailability (systemic absorption) of the composition disclosed herein is between 0.01 nM to 5 nM of ruxolitinib (for 1.5% of ruxolitinib, twice daily administration).

In another embodiment, the median bioavailability (systemic absorption) of the composition disclosed herein is between 0.01 nM to 5 nM of ruxolitinib (for 0.75% of ruxolitinib, twice daily administration).

In another embodiment, the median bioavailability (systemic absorption) of the composition disclosed herein is between 0.01 nM to 4 nM of ruxolitinib (for 1.5% of ruxolitinib, twice daily administration).

As used herein, the term “micronized” refers to ruxolitinib having a D90 particle size of between 1-100 microns.

In some embodiments, the ruxolitinib, or deuterated ruxolitinib or a pharmaceutically acceptable salt thereof is not dissolved or partially dissolved in the composition. “Partially dissolved” is used herein as up to 10%, 20%, 30%, 40%, 50% by weight solubility of the ruxolitinib, or deuterated ruxolitinib or a pharmaceutically acceptable salt within the composition.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

### Preparation of Water Suspension Gel of Ruxolitinib Phosphate, 2% (the 2% Refers to the Concentration of the Free Base Ruxolitinib)

In order to prepare a ruxolitinib phosphate water suspension gel, the following steps are performed:
a. 772 g aqueous buffer at pH 9, 20 g Tween 80 and 208 g ruxolitinib phosphate are milled with a WAB Dynomill for 25 min, while cooling.
b. 1 g of ethylenediaminetetraacetic acid disodium salt (EDTA), 5 g of Imidurea and 5 g of methylparaben (preservatives) are added to 125 g of milled ruxolitinib phosphate suspension.
c. 50 g of glycerin are dissolved in 900 kg of water. 10 of Carbopol 980 NF are added and the solution is mixed for 1 hour.
d. Sodium hydroxide, 20% solution is added slowly to form a gel.

### Example 2

### Encapsulation of Ruxolitinib Phosphate 15% (Corresponding to Free Base Ruxolitinib) Suspension

In order to prepare an encapsulated ruxolitinib phosphate, the following steps are performed:
a. Three solutions are prepared:
   i. An acid solution of 7% w/w citric acid, 10% w/w lactic acid and 32% w/w hydrochloric acid.
   ii. 3% Polyquaternium-7 aqueous solution (3 wt. %); and
   iii. 27% Sodium silicate solution
b. Ruxolitinib phosphate dispersion is prepared by mixing 15.6 g of ruxolitinib phosphate with 0.4 g CTAC (cetyltrimethylammonium chloride) and 45 g of aqueous buffer at pH 9, under high shear.
c. Preparation of encapsulated ruxolitinib phosphate:
   i. Sodium silicate (solution iii) is added to the ruxolitinib phosphate dispersion (dispersion of step b.) under high shear, followed by adding the acid solution (solution i) to adjust the pH to be about 9, and followed by addition of Polyquaternium-7 aqueous solution (solution ii) to the mixture.
   ii. The cycle is repeated 3-10 times at a temperature of between 28° C. to 40° C.
   iii. After the final cycle, the pH of the mixture is adjusted to about 9.0 using the acid solution, and water was added to complete the total weight of the mixture to 80 g.

### Example 3

### Water Suspension Gel of Encapsulated Ruxolitinib Phosphate, 2.5%

In order to prepare a water suspension gel of encapsulated ruxolitinib phosphate, the following steps are performed:
a. 1 g of ethylenediaminetetraacetic acid disodium salt (EDTA), 5 g of Imidurea and 5 g of methylparaben (preservatives) are added to 156.25 g of encapsulated ruxolitinib phosphate suspension as prepared in Example 2.
b. 50 g of glycerin are dissolved in 900 kg of water. 10 of Carbopol 980 NF are added and the solution is mixed for 1 hour.
c. Sodium hydroxide, 20% solution is added slowly to form a gel.

1. A regimen for the treatment, prevention or alleviation of an inflammatory skin condition comprising topically applying onto an affected skin area of a subject in need thereof, once a day, a topical composition which comprises more than 1.5% w/w and less than 3.0% w/w ruxolitinib, deuterated ruxolitinib or a pharmaceutically acceptable salt thereof and a pharmaceutically acceptable carrier.

2. The regimen of claim 1, wherein the treatment comprises a combination treatment with at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof.

3. The regimen of claim 1, wherein the regimen provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition.

4. (canceled)

5. The regimen of claim 1, wherein the ruxolitinib or pharmaceutically acceptable salt thereof, or deuterated ruxolitinib or a pharmaceutically acceptable salt thereof is in an amount of from about 2.0% w/w to about 2.5% w/w.

6. The regimen of claim 1, wherein the ruxolitinib or pharmaceutically acceptable salt thereof, or deuterated ruxolitinib or a pharmaceutically acceptable salt thereof is in an amount of about 2.5% w/w.

7. The regimen of claim 1, wherein the composition comprises ruxolitinib.

8. The regimen of claim 1, wherein the composition comprises deuterated ruxolitinib.

9. The regimen of claim 1, wherein the composition comprises ruxolitinib phosphate.

10. The regimen of claim 1, wherein the ruxolitinib or pharmaceutically acceptable salt thereof, or deuterated ruxolitinib or a pharmaceutically acceptable salt thereof is encapsulated.

11. The regimen of claim 1, wherein the ruxolitinib or pharmaceutically acceptable salt thereof, or deuterated ruxolitinib or a pharmaceutically acceptable salt thereof is not dissolved or partially dissolved in the composition.

12. The regimen of claim 1, wherein the ruxolitinib or pharmaceutically acceptable salt thereof, or deuterated ruxolitinib or a pharmaceutically acceptable salt thereof is micronized.

13. The regimen of claim 1, wherein the composition or the second composition is formulated as a cream, a gel, an ointment, an emulsion, a solution, a suspension, an elixir, a lotion, a tincture, a paste, a foam, an aerosol, a spray, a patch, a transdermal patch or an applicator syringe.

14. The regimen of claim 1, wherein the composition has decreased skin permeability, no systemic absorption or low systemic absorption.

15. The regimen of claim 1, wherein the composition has decreased skin permeability with a skin penetration lag time being between 2 hours to 24 hours.

16. The regimen of claim 1, wherein the composition has minor or negligible permeation capacity through the skin within the first hour of administration.

17. The regimen of claim 1, wherein said inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis.

18. The regimen of claim 17, wherein the inflammatory skin condition is atopic dermatitis.

19. A method of treatment, prevention or alleviation of an inflammatory skin condition, wherein the method comprises administering to a subject in need thereof, once a day, a topical composition a topical composition which comprises more than 1.5% w/w and less than 3.0% w/w ruxolitinib or pharmaceutically acceptable salt thereof, or deuterated ruxolitinib or a pharmaceutically acceptable salt thereof and a pharmaceutically acceptable carrier.

20. The method of claim 19, wherein the method comprises further administering at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof and a pharmaceutically acceptable carrier.

21. The method of claim 19, wherein the method provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition.

22. The method of claim 19, wherein the inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis.

23. The method of claim 22, wherein the inflammatory skin condition is atopic dermatitis.

24. (canceled)

25. The method of claim 19, wherein the ruxolitinib or pharmaceutically acceptable salt thereof, or deuterated ruxolitinib or a pharmaceutically acceptable salt thereof is in an amount of from about 2.0% w/w to about 2.5% w/w.

26. The method of claim 19, wherein the ruxolitinib or pharmaceutically acceptable salt thereof, or deuterated ruxolitinib or a pharmaceutically acceptable salt thereof is in an amount of about 2.5% w/w.

27. The method of claim 19, wherein the composition comprises ruxolitinib.

28. The method of claim 19, wherein the composition comprises deuterated ruxolitinib.

29. The method of claim 19, wherein the ruxolitinib or pharmaceutically acceptable salt thereof, or deuterated ruxolitinib or a pharmaceutically acceptable salt thereof is not dissolved or partially dissolved within the composition.

30. The method of claim 19, wherein the ruxolitinib or pharmaceutically acceptable salt thereof, or deuterated ruxolitinib or a pharmaceutically acceptable salt thereof is micronized.

31. The method of claim 19, wherein the composition comprises ruxolitinib phosphate.

32. The method of claim 19, wherein the composition has decreased skin permeability, no systemic absorption or low systemic absorption.

33. The method of claim 19, wherein the composition has decreased skin permeability with a skin penetration lag time being between 2 hours to 24 hours.

34. The method of claim 19, wherein the composition has minor or negligible permeation capacity through the skin within the first hour of administration.

35. The method of claim 19, wherein the ruxolitinib or pharmaceutically acceptable salt thereof, or deuterated ruxolitinib or a pharmaceutically acceptable salt thereof is encapsulated.

36. The method of claim 19, wherein the composition is formulated as a cream, a gel, an ointment, an emulsion, a solution, a suspension, an elixir, a lotion, a tincture, a paste, a foam, an aerosol, a spray, a patch, a transdermal patch or an applicator syringe.

37. A regimen for the treatment, prevention or alleviation of an inflammatory skin condition comprising topically applying onto an affected skin area of a subject in need thereof, at least once a day, a topical composition which comprises more than 0.5% w/w and less than 3.0% w/w ruxolitinib, deuterated ruxolitinib or a pharmaceutically acceptable salt thereof and a pharmaceutically acceptable carrier, wherein the composition has decreased skin permeability, no systemic absorption or low systemic absorption.

38. The regimen of claim 37, wherein the topical composition is administered twice daily or three times per day.

39. The regimen of claim 37, wherein the treatment comprises a combination treatment with at least one additional active agent selected from about 0.01% w/w to about 0.25% w/w a low-dose steroid, from about 0.25% w/w to about 3% w/w roflumilast, from about 0.25% w/w to about 3.0% w/w tapinarof and any combination thereof.

40. The regimen of claim 37, wherein topically applying onto an affected skin area of a subject in need thereof, once a day, provides a comparable or lower side effects compared to a twice daily of a systemic administration; or comparable or lower side effects compared to twice daily of a topical administration of a corresponding ruxolitinib, deuterated ruxolitinib, or pharmaceutically acceptable salt thereof composition.

41. (canceled)

42. The regimen of claim 37, wherein the ruxolitinib or pharmaceutically acceptable salt thereof, or deuterated ruxolitinib or a pharmaceutically acceptable salt thereof is in an amount of from about 0.5% w/w to about 1.5% w/w.

43. The regimen of claim 37, wherein the composition comprises ruxolitinib.

44. The regimen of claim 37, wherein the composition comprises deuterated ruxolitinib.

45. The regimen of claim 37, wherein the composition comprises ruxolitinib phosphate.

46. The regimen of claim 37, wherein the ruxolitinib or pharmaceutically acceptable salt thereof, or deuterated ruxolitinib or a pharmaceutically acceptable salt thereof is encapsulated.

47. The regimen of claim 37, wherein the ruxolitinib or pharmaceutically acceptable salt thereof, or deuterated ruxolitinib or a pharmaceutically acceptable salt thereof is micronized.

48. The regimen of claim 37, wherein the composition or the second composition is formulated as a cream, a gel, an ointment, an emulsion, a solution, a suspension, an elixir, a lotion, a tincture, a paste, a foam, an aerosol, a spray, a patch, a transdermal patch or an applicator syringe.

49. The regimen of claim 37, wherein said inflammatory skin condition is selected from the group consisting of: acne, rosacea, atopic dermatitis, psoriasis, flexural/inverse psoriasis, eczema, contact dermatitis, urticaria, dermatitis herpetiformis, lichen planus and seborrheic dermatitis.

50. The regimen of claim 49, wherein the inflammatory skin condition is atopic dermatitis.

51. A method of treatment, prevention or alleviation of an inflammatory skin condition, wherein the method comprises administering to a subject in need thereof, once or twice or three times a day, a topical composition a topical composition which comprises more than 0.5% w/w and less than 3.0% w/w ruxolitinib or pharmaceutically acceptable salt thereof, or deuterated ruxolitinib or a pharmaceutically acceptable salt thereof and a pharmaceutically acceptable carrier, wherein the composition has decreased skin permeability, no systemic absorption or low systemic absorption.

52. The method of claim 51, wherein the composition has decreased skin permeability with a skin penetration lag time being between 2 hours to 24 hours.

53. The method of claim 51, wherein the composition has minor or negligible permeation capacity through the skin within the first hour of administration.
